SEQUENCE LISTING (65 sequences)

SEQ ID NO: 1 - protein, 30 aa, synthetic construct
DAAWGGGLLMSRKHKWKLSGVERANSVTWN

SEQ ID NO: 2 - protein, 15 aa, synthetic construct
DAAWGGGLLMSRKHK

SEQ ID NO: 3 - protein, 15 aa, synthetic construct
WGGGLLMSRKHKWKL

SEQ ID NO: 4 - protein, 15 aa, synthetic construct
GLLMSRKHKWKLSGV

SEQ ID NO: 5 - protein, 15 aa, synthetic construct
MSRKHKWKLSGVERA

SEQ ID NO: 6 - protein, 15 aa, synthetic construct
KHKWKLSGVERANSV

SEQ ID NO: 7 - protein, 15 aa, synthetic construct
WKLSGVERANSVTWN

SEQ ID NO: 8 - protein, 9 aa, synthetic construct
KHKWKLSGV

SEQ ID NO: 9 - protein, 10 aa, synthetic construct
KHKWKLSGVE

SEQ ID NO: 10 - protein, 11 aa, synthetic construct
KHKWKLSGVER

SEQ ID NO: 11 - protein, 12 aa, synthetic construct
KHKWKLSGVERA

SEQ ID NO: 12 - protein, 13 aa, synthetic construct
KHKWKLSGVERAN

SEQ ID NO: 13 - protein, 14 aa, synthetic construct
KHKWKLSGVERANS

SEQ ID NO: 14 - protein, 14 aa, synthetic construct
HKWKLSGVERANSV

SEQ ID NO: 15 - protein, 13 aa, synthetic construct
KWKLSGVERANSV

SEQ ID NO: 16 - protein, 12 aa, synthetic construct
WKLSGVERANSV

SEQ ID NO: 17 - protein, 11 aa, synthetic construct
KLSGVERANSV

SEQ ID NO: 18 - protein, 10 aa, synthetic construct
LSGVERANSV

SEQ ID NO: 19 - protein, 9 aa, synthetic construct
SGVERANSV

SEQ ID NO: 20 - protein, 8 aa, synthetic construct
WKLSGVER

SEQ ID NO: 21 - protein, 9 aa, synthetic construct
WKLSGVERA

SEQ ID NO: 22 - protein, 14 aa, synthetic construct
KKKWKLSGVERKKK

SEQ ID NO: 23 - protein, 15 aa, synthetic construct
KKKWKLSGVERAKKK

SEQ ID NO: 24 - protein, 31 aa, synthetic construct
IFSPGGAISNMYAMMIARFKMFPEVKEKGMA

SEQ ID NO: 25 - protein, 15 aa, synthetic construct
IFSPGGAISNMYAMM

SEQ ID NO: 26 - protein, 15 aa, synthetic construct
PGGAISNMYAMMIAR

SEQ ID NO: 27 - protein, 15 aa, synthetic construct
AISNMYAMMIARFKM

SEQ ID NO: 28 - protein, 15 aa, synthetic construct
NMYAMMIARFKMFPE

SEQ ID NO: 29 - protein, 15 aa, synthetic construct
AMMIARFKMFPEVKE

SEQ ID NO: 30 - protein, 16 aa, synthetic construct
IARFKMFPEVKEKGMA

SEQ ID NO: 31 - protein, 14 aa, synthetic construct
ISNMYAMMIARFKM

SEQ ID NO: 32 - protein, 13 aa, synthetic construct
SNMYAMMIARFKM

SEQ ID NO: 33 - protein, 12 aa, synthetic construct
NMYAMMIARFKM

SEQ ID NO: 34 - protein, 11 aa, synthetic construct
MYAMMIARFKM

SEQ ID NO: 35 - protein, 10 aa, synthetic construct
YAMMIARFKM

SEQ ID NO: 36 - protein, 9 aa, synthetic construct
AMMIARFKM

SEQ ID NO: 37 - protein, 14 aa, synthetic construct
AISNMYAMMIARFK

SEQ ID NO: 38 - protein, 13 aa, synthetic construct
AISNMYAMMIARF

SEQ ID NO: 39 - protein, 12 aa, synthetic construct
AISNMYAMMIAR

SEQ ID NO: 40 - protein, 11 aa, synthetic construct
AISNMYAMMIA

SEQ ID NO: 41 - protein, 10 aa, synthetic construct
AISNMYAMMI

SEQ ID NO: 42 - protein, 9 aa, synthetic construct
AISNMYAMM

SEQ ID NO: 43 - protein, 19 aa, synthetic construct
KKKRISNMYAMMIARRKKK

SEQ ID NO: 44 - protein, 19 aa, synthetic construct
KKKKISNMYAMMIARKKKK

SEQ ID NO: 45 - protein, 10 aa, synthetic construct
ISNMYAMMIA

SEQ ID NO: 46 - protein, 30 aa, synthetic construct
DLERRILEAKQKGFVPFLVSATAGTTVYGA

SEQ ID NO: 47 - protein, 15 aa, synthetic construct
DLERRILEAKQKGFV

SEQ ID NO: 48 - protein, 15 aa, synthetic construct
RRILEAKQKGFVPFL

SEQ ID NO: 49 - protein, 15 aa, synthetic construct
LEAKQKGFVPFLVSA

SEQ ID NO: 50 - protein, 15 aa, synthetic construct
KQKGFVPFLVSATAG

SEQ ID NO: 51 - protein, 15 aa, synthetic construct
GFVPFLVSATAGTTV

SEQ ID NO: 52 - protein, 17 aa, synthetic construct
LERRILEAKQKGFVPFL

SEQ ID NO: 53 - protein, 16 aa, synthetic construct
ERRILEAKQKGFVPFL

SEQ ID NO: 54 - protein, 15 aa, synthetic construct
RRILEAKQKGFVPFL

SEQ ID NO: 55 - protein, 14 aa, synthetic construct
RILEAKQKGFVPFL

SEQ ID NO: 56 - protein, 13 aa, synthetic construct
ILEAKQKGFVPFL

SEQ ID NO: 57 - protein, 12 aa, synthetic construct
LEAKQKGFVPFL

SEQ ID NO: 58 - protein, 17 aa, synthetic construct
DLERRILEAKQKGFVPF

SEQ ID NO: 59 - protein, 16 aa, synthetic construct
DLERRILEAKQKGFVP

SEQ ID NO: 60 - protein, 15 aa, synthetic construct
DLERRILEAKQKGFV

SEQ ID NO: 61 - protein, 14 aa, synthetic construct
DLERRILEAKQKGF

SEQ ID NO: 62 - protein, 13 aa, synthetic construct
DLERRILEAKQKG

SEQ ID NO: 63 - protein, 12 aa, synthetic construct
DLERRILEAKQK

SEQ ID NO: 64 - protein, 18 aa, synthetic construct
KKERRILEAKQKGFVPKK

SEQ ID NO: 65 - protein, 14 aa, synthetic construct
ERRILEAKQKGFVP

(19) United States
(12) Patent Application Publication
(10) Pub. No.: US 2026/0034200 A1
(43) Pub. Date: Feb. 5, 2026

(54) TOLEROGENIC PEPTIDES

(71) Applicant: University of Birmingham, Birmingham (GB)

(72) Inventors: David Cameron Wraith, Birmingham (GB); Parth Narendran, Birmingham (GB); Tien Haeng Ng, Birmingham (GB); Michael John Price, Birmingham (GB); Alastair Copland, Birmingham (GB)

(21) Appl. No.: 19/099,041

(22) PCT Filed: Jul. 27, 2023
(86) PCT No.: PCT/GB2023/051990

§ 371 Date: Jan. 27, 2025

(30) Foreign Application Priority Data
Jul. 27, 2022 (GB) 2210967.2

(51) Int. Cl. A61K 39/00; A61P 3/10; A61P 37/06; C12N 9/88

(52) U.S. Cl. CPC A61K 39/0008; A61P 3/10; A61P 37/06; C12N 9/88; C12Y 401/01015

(57) ABSTRACT

The present disclosure is based in part on studies on novel tolerogenic peptides derived from a protein expressed by a pancreatic cell, which have been developed for use in antigen-specific immunotherapy for type 1 diabetes. Disclosed is a tolerogenic peptide capable of binding an MHC class II molecule independent of antigen processing for use in the treatment of type 1 diabetes, wherein the peptide is derived from a protein expressed by a pancreatic cell.

### FIELD

The present disclosure relates to tolerogenic peptides and uses thereof in the treatment or prevention of autoimmune disease.

### BACKGROUND

Type 1 diabetes is a chronic autoimmune condition resulting from the destruction of insulin secreting pancreatic beta cells (Atkinson et al., 2014). This is evidenced by studies in mouse models where type 1 diabetes can be recapitulated through transfer of T-cell clones specific to beta cell proteins (Peterson and Haskins, 1996). Similarly, in humans, type 1 diabetes pancreas is characterised by the infiltration of CD8 and CD4 T-cells which target specific beta cell derived proteins (Babon et al., 2016). Whilst therapy for type 1 diabetes exists in the form of insulin injection therapy, titrating insulin replacement therapy to reproduce a physiological insulin secretion profile is challenging and imperfect. Alternative therapeutic strategies attempt to modulate the function of beta cell antigen-specific T-cells to delay the progression of type 1 diabetes. Early trials with non-specific immunosuppressive agents resulted in improved diabetes control (Feutren et al., 1986). More recent studies where T-cells have been targeted with non-antigen-specific immunomodulatory monoclonal antibodies to CD3 have demonstrated a preservation of beta cell function in patients with new onset type 1 diabetes, where there is residual beta cell function to preserve, which has led to recent US FDA approval (Herold et al. 2019, Mullard 2022). However, non-antigen-specific approaches come with the risk of side effects associated with systemic immunosuppression-such as the re-activation of latent viral infections in T1D patients after B cell depletion (Kroll et al., 2013). Thus, there is a need for the development of more selective immunosuppressive therapies that target beta cell specific autoreactive T-cells.

Previous studies have shown that injecting animal models with accurately identified T-cell epitopes can generate T-cells that are capable of migrating to the target organ, supress the autoimmune process at that site, and modulate the onset of clinical disease (Burton et al., 2014). Various immunomodulatory peptides have been disclosed in the art for use in the treatment of autoimmune diseases, such as type 1 diabetes. WO1994004557A1 discloses immunomodulatory peptides comprising a segment of a naturally-occurring human protein that bind to a human major histocompatibility complex class II allotype. US20070026465A1 discloses a method of detecting GAD65 specific T-cells for the prediction of type 1 diabetes onset. US20040234531A1 discloses a chimeric molecule comprising an IILA-DR element linked to an epitope of GAD65, an antigen associated with autoimmune diabetes, stimulated the secretion of the inhibitory cytokine IL-10 from CD4 T-cells of type I diabetic patients. US20090305340A1 discloses modified GAD65 compositions that antagonise the activities of islet-specific T-cells that contribute to the progression of one or more autoimmune disorders. GB2454687A discloses MHC class II binding peptides derived from GAD65, wherein these peptides may be used to produce MHC-peptide complexes, or to generate antibodies or CD4+ cell lines specific to these peptides. However, not all T-cell epitopes derived from an antigen will induce tolerance in autoreactive T-cells to self-antigens. For use of peptides in antigen-specific immunotherapy, it is critical to develop peptides that accurately mimic a naturally processed antigen, such that the peptides are able to bind MHC class II molecules without further antigen processing and engage self-antigen reactive T-cells.

It is amongst the objectives of the present disclosure to develop novel tolerogenic peptides that would mitigate one or more of the aforementioned disadvantages of existing treatments for type 1 diabetes.

### SUMMARY

The present disclosure is based in part on studies on novel tolerogenic peptides derived from a protein expressed by a pancreatic cell, which have been developed for use in antigen-specific immunotherapy for type 1 diabetes.

In a first aspect, there is provided a tolerogenic peptide capable of binding an MHC class II molecule independent of antigen processing for use in the treatment of type 1 diabetes, wherein the peptide is derived from a protein expressed by a pancreatic cell.

Type 1 diabetes is a chronic autoimmune disease that is primarily mediated by the destruction of pancreatic beta cells by autoreactive CD4 and CD8 T-cells. Treatment of type 1 diabetes typically comprises insulin replacement therapy, and optionally other treatment methods that attempt to restore beta cell function or regulate the immune response in a non-specific manner to prevent the destruction of beta cells. However, there is a need for development of immunotherapies that selectively target self-reactive immune cells while not affecting other aspects of immune function.

The principle underlying antigen-specific immunotherapy is that exposure to an antigen in an environment that is tolerogenic (rather than inflammatory) will educate T-cells to downregulate (rather than activate) subsequent T-cell responses to that antigen. The downregulated T-cell response includes the secretion of anti-inflammatory cytokines (Burton et al., 2014), which can act locally to downregulate other T-cells in the vicinity—a concept recognised as bystander suppression (Wraith, 2016). The inventors have developed novel tolerogenic peptides derived from GAD65 that bind major histocompatibility complex (MHC) class II molecules independent of antigen processing and induce T-cell mediated immune tolerance. Importantly, these tolerogenic peptides of the present disclosure bind to MHC class II molecules of antigen presenting cells without the need for further antigen processing. If a peptide is too long to bind the peptide binding groove of an MHC molecule without further processing, or binds in an inappropriate conformation, then it will not be tolerogenic in vivo. As not all T cell epitopes induce tolerance to self-antigens, it is critical to identify specific T-cell epitopes that mimic the conformation of a naturally processed antigen capable of being presented on an MHC class II molecule for downregulation of autoreactive CD4+ T-cells.

The term “tolerogenic” as used herein means capable of inducing tolerance to a particular antigen. Immune tolerance refers to a range of host processes that prevent potentially harmful immune responses and results in a state of unresponsiveness of the immune system to substances or tissue(s) that have the capacity to elicit an immune response. Immune tolerance is a highly regulated process that enables the discrimination of self from non-self, suppression of allergic responses and prevention of reactive immune responses towards foetal antigens by the maternal immune system.

Self-tolerance is the ability to prevent an immune response to self-produced antigens. If the immune system elicits an aberrant immune response to self-antigens, an autoimmune disease may result. For example, in autoimmune conditions such as type 1 diabetes, the individual lacks immune tolerance to their own self antigens and the body does not discriminate correctly between self and non-self antigens.

We envisage that it may be possible to induce immunological tolerance towards specific T-cell epitopes by administration of soluble peptide analogues. In a normal adaptive immune response, T-cells recognise internal epitopes of a protein antigen. Antigen presenting cells (APCs) internalise protein antigens and degrade them into short fragments (antigen processing). A peptide may bind a MHC class I or II molecule inside the cell for presentation at the cell surface. The peptide presented on an MHC molecule may be recognised by a T-cell, in which case the peptide is a T-cell epitope. Such epitopes in peptide form can be used to induce immunological tolerance, which are referred to as tolerogenic peptides throughout this disclosure.

Tolerance may result from or be characterised by the induction of anergy in at least a portion of CD4+ T-cells. In order to activate a T-cell, a peptide must associate with a professional APC capable of delivering two signals to T-cells. The first signal is delivered by the MHC-peptide complex on the cell surface of the APC and is received by the T-cell via the T-cell receptor.

The second signal is delivered by costimulatory molecules on the surface of the APC, such as CD80 and CD86, and received by CD28 on the surface of the T-cell. It is thought that when a T-cell receives the first signal in the absence of the second signal, it is not activated and becomes anergic. Anergic T-cells are refractory to subsequent antigenic challenge, and may be capable of suppressing other immune responses. Anergic T-cells are thought to be involved in mediating T-cell tolerance. Peptides that are capable of binding class II MHC molecules without antigen processing will be able to bind MHC molecules on immature APCs. Thus, they are likely to be presented to T-cells without co-stimulation, leading to T-cell anergy and tolerance.

Peptides which require processing before they can be presented in conjunction with MHC molecules do not induce tolerance because they have to be processed by mature APCs. Mature APCs (e.g. macrophages, B cells and dendritic cells) are capable of antigen processing, but also of delivering both first and second signals to a T-cell, resulting in T-cell activation. It is envisaged that the peptides of the correct size and conformation capable of binding an MHC molecule independent of antigen processing, such as those described herein, are likely to bind MHC molecules on immature APCs and induce T-cell anergy.

The term “peptide” as used herein may refer to a fragment of wild-type or reference sequence, such as a fragment up to 40 amino acids in length. In one embodiment, peptides as described herein for use in treatment of type 1 diabetes may comprise a fragment of wild-type or reference sequence which is at least 5 amino acids in length. In some embodiments, peptides as described herein may be between 5 to 40 amino acids in length. In some embodiments, peptides as described herein may be between 5 to 20, 25, 30 or 35 amino acids in length. In any of these embodiments, the peptide is derived from GAD65 and comprises at least a minimal epitope. An epitope is a peptide derivable from an antigen which is capable of binding to the peptide-binding groove of an MHC class I or II molecule and being recognised by a T cell. The minimal epitope is a peptide derivable from an antigen which is capable of binding to the peptide-binding groove of an MHC class I or II molecule and being recognised by a T-cell.

The term “protein expressed by a pancreatic cell” refers to any protein that is expressed by a pancreatic cell and found to be expressed intracellularly and/or may be membrane-bound. The protein may be differentially expressed (such as over or underexpressed) in a pancreatic cell from a subject or by subjects with type-1 diabetes, as compared to the same pancreatic cell from a subject without type-1 diabetes. In some instances, the protein expressed by a pancreatic cell may refer to proteins that are synthesised by a pancreatic cell for secretion. The term “a pancreatic cell”, refers to any cell found within the pancreas, such as an alpha or beta cell found in pancreatic islets, for example.

In one embodiment, the tolerogenic peptide derived from a protein expressed by a pancreatic cell may comprise a fragment of glutamate decarboxylase 65 (GAD65) or a variant thereof.

In one teaching, the disclosure provides a tolerogenic peptide that must bind an MHC class II molecule without further antigen processing, wherein the peptide comprises a fragment of GAD65, and the presentation of the tolerogenic peptide-MHC class II complex to a CD4+ T-cell elicits a tolerogenic response. In one embodiment, the tolerogenic peptide may comprise a fragment of a wild-type or reference GAD65 sequence of at least 5 amino acids in length. In one embodiment, the tolerogenic peptide may comprise a fragment of GAD65 comprising 5 to 40 amino acids in length. In some embodiments, the fragment may be 5 to 20, 25, 30 or 35 amino acids in length.

The wild-type or reference sequence of GAD65 from which the tolerogenic peptide may correspond to the species of the subject to which the tolerogenic peptide is being provided. In one embodiment, the preferred wild-type or reference GAD65 sequence from which the tolerogenic peptide is derived is mammalian, such as a human GAD65 sequence. In an alternative embodiment, the wild-type or reference GAD65 sequences from which the tolerogenic peptide is derived may be from mouse, rat, dog, cat, pig, sheep or horse GAD65 sequence, for example.

“Peptide” as used herein may also encompass variants of the fragment(s) of the wild-type or reference sequence, wherein the variant may comprise one or more amino acid modifications. In one embodiment, the variant may comprise at most 20, 15, 10, 9, 8, 7, 6, 5, 4, 3 or 2 modifications. In one embodiment, the variant may comprise at least 2, 3, 4 or 5 modifications. The term “peptide” as used herein encompasses peptides obtained from peptides derived from naturally occurring proteins or may be synthetically synthesised peptides. The term “peptide derived from a protein” refers to a peptide obtained by cleavage of a naturally occurring protein.

In some embodiments, the tolerogenic peptide may comprise a variant peptide that exhibits at least 80%, 85%, 86%, 87%, 88%, 89%, 90%, 91%, 92%, 93%, 94%, 95%, 96%, 97%, 98% or 99% sequence identity to the sequence found in the wild-type or reference sequence of GAD65. Any of the peptides of GAD65 as described herein that is capable of binding an MHC class II molecule independent of antigen processing and induces immune tolerance may be provided for use in the treatment of type 1 diabetes.

An amino acid modification may refer to a substitution of a wild-type or reference amino acid with another. Such substitutions may be conservative in that they swap a wild-type residue for another with the same or similar structural, chemical and/or physio-chemical properties. “Conservative” amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Naturally occurring peptides or proteins comprise amino acids in the L-configuration. In some instances, the amino acid modification of the present disclosure may comprise a substitution with an equivalent amino acid in the D-configuration or a conservative amino acid substitution with a D-amino acid. In other instances, the amino acid modification may comprise substitution with an unusual amino acid provided that the tolerogenic peptide retains its ability to bind an MHC class II molecule independent of antigen processing, such as citrulline, hydroxyproline, beta-alanine, ornithine, norleucine, 3-nitrotyrosine, pyroglutamic acid, nitroarginine, for example. Alternatively, the peptide sequence may comprise modified amino acids, such as homo-amino acids, beta-homo-amino acids, N-methyl amino acids and/or alpha-methyl amino acids.

Substitutions may also be ‘non-conservative’ in that a wild-type residue is substituted for an amino acid of a different class, for example an amino acid which is structurally dissimilar, chemically different and/or physio-chemically different or dissimilar.

An amino acid modification may comprise the deletion of an amino acid residue from a wild-type or reference sequence. Other amino acid modifications may comprise the insertion of one or more amino acids into a wild-type or reference sequence. Amino acid modifications may further comprise the inversion of certain parts or portions of the wild-type/reference sequence.

Optionally, or in addition to the abovementioned modifications, the peptides as described herein may be modified accordingly by the skilled person using methods known in the art to alter one of more pharmacokinetic properties of the tolerogenic peptide, for example. In one embodiment, the tolerogenic peptide may be modified in order to increase its the solubility. Hydrophilic lysine residues may be subsequently added to a peptide to increase the core epitope solubility. In one embodiment, the amino acid modification may comprise one or more amino acid modifications that increase the solubility of the peptide, such as addition or substitution of a wild-type or reference residue with one or more amino acids selected from lysine, arginine, histidine, aspartic acid, glutamic acid, serine, threonine, asparagine and/or glutamine. In some embodiments, the amino acid modification for increasing solubility of any one or more tolerogenic peptides as disclosed herein may comprise addition or substitution of a wild-type residue with lysine. In one embodiment, the modified tolerogenic peptide with improved solubility has a grand average of hydropathy (GRAVY) score lower than 0.

The peptide may also demonstrate good bioavailability in vivo. The peptide may maintain a conformation in vivo which enables it to bind to an MHC molecule at the cell surface without due hindrance.

By way of summary, the tolerogenic peptides may comprise any one or more of the disclosed sequences in Tables 1-3. In one embodiment, the tolerogenic peptide for use in the treatment of type 1 diabetes comprises, consists essentially of, or consists of the sequence identified as the “minimal” peptide sequence in any one of the Tables 1 to 3, wherein the peptide may additionally comprise one or more further modifications according to any modifications described herein. “Consists essentially of” or “consists of” means that any of the GAD65-derived peptide sequences as disclosed herein may contain additional N- and/or C-terminally located stretches of amino acids that are not necessarily forming part of the peptide that functions as the core epitope sequence, provided that the peptide retains its ability to bind an MHC class II molecule independent of antigen processing.

TABLE 1
GAD65-derived peptide sequence set I

| Peptide | Sequence | SEQ ID NO |
|---|---|---|
| P10 30mer | DAAWGGGLLMSRKHK | 1 |
| | WKLSGVERANSVTWN | |
| P10.1 15mer | DAAWGGGLLMSRKHK | 2 |
| P10.2 15mer | WGGGLLMSRKHKWKL | 3 |
| P10.3 15mer | GLLMSRKHKWKLSGV | 4 |
| P10.4 15mer | MSRKHKWKLSGVERA | 5 |
| P10.5 15mer | KHKWKLSGVERANSV | 6 |
| P10.6 15mer | WKLSGVERANSVTWN | 7 |
| P10.5 R6 | KHKWKLSGV | 8 |
| P10.5 R5 | KHKWKLSGVE | 9 |
| P10.5 R4 | KHKWKLSGVER | 10 |
| P10.5 R3 | KHKWKLSGVERA | 11 |
| P10.5 R2 | KHKWKLSGVERAN | 12 |
| P10.5 R1 | KHKWKLSGVERANS | 13 |
| P10.5 L1 | HKWKLSGVERANSV | 14 |
| P10.5 L2 | KWKLSGVERANSV | 15 |
| P10.5 L3 | WKLSGVERANSV | 16 |
| P10.5 L4 | KLSGVERANSV | 17 |
| P10.5 L5 | LSGVERANSV | 18 |
| P10.5 L6 | SGVERANSV | 19 |
| P10.5.C | WKLSGVER | 20 |
| (Minimal) | | |
| P10.5.C+1 | WKLSGVERA | 21 |
| P10.5.C.6K | KKKWKLSGVERKKK | 22 |
| P10.5.C+1.6K | KKKWKLSGVERAKKK | 23 |
| (P10Sol) | | |

TABLE 2
GAD65-derived peptide sequence set II

| Peptide | Sequence | SEQ ID NO |
|---|---|---|
| P5 30mer | IFSPGGAISNMYAMM | 24 |
| | IARFKMFPEVKEKGMA | |
| P5.1 | IFSPGGAISNMYAMM | 25 |
| P5.2 | PGGAISNMYAMMIAR | 26 |
| P5.3 | AISNMYAMMIARFKM | 27 |
| P5.4 | NMYAMMIARFKMFPE | 28 |
| P5.5 | AMMIARFKMFPEVKE | 29 |
| P5.6 | IARFKMFPEVKEKGMA | 30 |
| P5.3L1 | ISNMYAMMIARFKM | 31 |
| P5.3L2 | SNMYAMMIARFKM | 32 |
| P5.3L3 | NMYAMMIARFKM | 33 |
| P5.3L4 | MYAMMIARFKM | 34 |
| P5.3L5 | YAMMIARFKM | 35 |
| P5.3L6 | AMMIARFKM | 36 |
| P5.3R1 | AISNMYAMMIARFK | 37 |
| P5.3R2 | AISNMYAMMIARF | 38 |
| P5.3R3 | AISNMYAMMIAR | 39 |
| P5.3R4 | AISNMYAMMIA | 40 |
| P5.3R5 | AISNMYAMMI | 41 |
| P5.3R6 | AISNMYAMM | 42 |
| P5.3.C+1.2R6K | KKKRISNMYAMMIARRKKK | 43 |
| P5.3.C+1.8K | KKKKISNMYAMMIARKKKK | 44 |
| P5.3.C | ISNMYAMMIA | 45 |
| (Minimal) | | |

TABLE 3
GAD65-derived peptide sequence set III

| Peptide | Sequence | SEQ ID NO |
|---|---|---|
| P8 30mer | DLERRILEAKQKGFVPFLVSATAGTTVYGA | 46 |
| P8.1 | DLERRILEAKQKGFV | 47 |
| P8.2 | RRILEAKQKGFVPFL | 48 |
| P8.3 | LEAKQKGFVPFLVSA | 49 |
| P8.4 | KQKGFVPFLVSATAG | 50 |
| P8.5 | GFVPFLVSATAGTTV | 51 |
| P8.1L1 | LERRILEAKQKGFVPFL | 52 |
| P8.1L2 | ERRILEAKQKGFVPFL | 53 |
| P8.1L3 | RRILEAKQKGFVPFL | 54 |
| P8.1L4 | RILEAKQKGFVPFL | 55 |
| P8.1L5 | ILEAKQKGFVPFL | 56 |
| P8.1L6 | LEAKQKGFVPFL | 57 |
| P8.2R1 | DLERRILEAKQKGFVPF | 58 |
| P8.2R2 | DLERRILEAKQKGFVP | 59 |
| P8.2R3 | DLERRILEAKQKGFV | 60 |
| P8.2R4 | DLERRILEAKQKGF | 61 |
| P8.2R5 | DLERRILEAKQKG | 62 |
| P8.2R6 | DLERRILEAKQK | 63 |
| P8.1.C+4K | KKERRILEAKQKGFVPKK | 64 |
| P8.1.C | ERRILEAKQKGFVP | 65 |
| (Minimal) | | |

In a certain embodiment, the tolerogenic peptide may comprise the amino acid sequence IFSPGGAISNMYAMMIARFKMFPEVKEKGMA, DLERRILEAKQKGFVPFLVSATAGTTVYGA or DAAWGGGLLMSRKHKWKLSGVERANSVTWN for use in the treatment of type 1 diabetes. In one embodiment, the tolerogenic peptide may consist essentially of a fragment derived from IFSPGGAISNMYAMMIARFKMFPEVKEKGMA, DLERRILEAKQKGFVPFLVSATAGTTVYGA or DAAWGGGLLMSRKHKWKLSGVERANSVTWN, wherein the fragment may comprise at least 5 amino acids in length, optionally modified, as described above, to alter one of more pharmacokinetic properties of the tolerogenic peptide. In one embodiment, the fragment may comprise between 5 to 20 amino acids in length.

In one embodiment, the tolerogenic peptide may be derived from glutamate decarboxylase 65 (GAD65) and comprise the sequence WKLSGVER, ISNMYAMMIA and/or ERRILEAKQKGFVP capable of binding an MHC class II molecule independent of antigen processing for use in the treatment of type 1 diabetes. In one embodiment, the said MHC class II molecule binding portion of the peptide derived from GAD65 may have a maximum length of 14 amino acids and/or optionally, the peptide further comprises up to 3 (such as 1, 2 or 3) additional amino acids at the N- or C-terminus, or at both termini, that modify one or more pharmacokinetic properties of the peptide, such as solubility of the peptide in an aqueous environment (e.g. blood).

Using any one or more of the peptides as described herein, the disclosure also provides a method of treating or preventing type 1 diabetes, said method comprising administering a subject in need thereof a therapeutically effective amount of any of the tolerogenic peptides as described herein. In an alternative embodiment, the said method may comprise administering a plurality of peptides described herein.

A subject to be administered a tolerogenic peptide of this disclosure may include any human or animal subject with type 1 diabetes. The subject may also be any human or animal subject predisposed and/or susceptible to developing type 1 diabetes, wherein type 1 diabetes may be treated, ameliorated, or prevented with the use of one or more tolerogenic peptides as described herein.

The present disclosure is also directed to the use of tolerogenic peptide(s) as described herein in the manufacture of a medicament for use in treating or preventing type 1 diabetes. The peptide(s) may be manufactured according to any method of peptide synthesis known in the art, such as liquid-phase peptide synthesis (LPPS) based techniques or solid-phase peptide synthesis (SPSS). For example, peptides can be synthesized by SPSS (Roberge J Y et al (1995) Science 269:202-204), cleaved from the resin, and purified by preparative high performance liquid chromatography (e.g., Creighton (1983) Proteins Structures And Molecular Principles, WH Freeman and Co, New York NY). Automated synthesis may be achieved, for example, using the ABI 43 1 A Peptide Synthesizer (Perkin Elmer) in accordance with the instructions provided by the manufacturer. The peptide may alternatively be made by recombinant means, or by cleavage from a longer polypeptide. For example, the peptide may be obtained by cleavage from the GAD65 protein, which may be followed by modification of one or both ends. The composition of a peptide may be confirmed by amino acid analysis or sequencing. The peptides may be manufactured with or subsequently combined with any substance(s) known in the art that increases stability and/or solubility of the peptide(s).

In one teaching, the tolerogenic peptides as described herein may be provided as a pharmaceutical composition, optimally formulated with at least one pharmaceutically acceptable excipient thereof. In one embodiment, an acceptable excipient may be selected from water, saline (e.g. phosphate-buffered saline), human serum albumin, dextrose, trehalose, sucrose, mannitol, sorbitol, polysorbate 20, polysorbate 80, glycerol, ethanol, polyethylene glycol, or the like and combinations thereof. The pharmaceutical composition may comprise a therapeutically or prophylactically effective amount of one or more tolerogenic peptides as described herein. In one embodiment, the pharmaceutical composition may comprise an effective amount of any one or more tolerogenic peptides of the present disclosure or a combination thereof.

An effective amount of a tolerogenic peptide refers to an amount of the peptide that is sufficient to induce immune tolerance or minimise and/or reduce autoreactive T-cell activation to an epitope and/or antigen of interest. Typically, the tolerogenic peptide is able to reduce autoreactive T-cell activation by at least 20%, such as 30%, 50%, 60% 70%, 80%, or more. It is possible to test this using tests described herein, as well as counting the number of T-cells or percentage of T-cells using techniques known in the art. Alternatively, a reduction in cytokine induction may be correlated with a reduction in T-cell activation. The tolerogenic peptides of the pharmaceutical composition are capable of binding MHC class II molecules independent of antigen processing. In some instances, the tolerogenic peptides may also bind MHC class I molecules.

In one embodiment, the pharmaceutical composition may optionally further comprise one or more pharmaceutically acceptable stabilisers, wetting agents, emulsifiers, salts, buffers and/or adjuvants known in the art.

The tolerogenic peptides may be formulated into the composition as non-ionic or salt forms. Pharmaceutically acceptable salt refers to a salt of a compound that is pharmaceutically acceptable and that possesses, or can be converted to a form that possesses, the desired pharmacological activity of the parent compound. Such salts include acid addition salts formed with inorganic acids, such as hydrochloric acid, sulphuric acid, nitric acid, phosphoric acid and the like; or formed with organic acids, such as acetic acid, citric acid, glucoheptonic acid, lactic acid, for example.

In one embodiment, there is provided a method for treatment of type 1 diabetes comprising administration of an effective amount of a tolerogenic peptide or a pharmaceutical composition thereof, as described herein, to a subject suffering from, or predisposed to developing, type 1 diabetes.

It may be possible to provide the tolerogenic peptide or pharmaceutical composition as a combination therapy in conjunction with other types of treatment for type 1 diabetes. In one embodiment, the tolerogenic peptide of the present disclosure may be combined with one or more active ingredients and/or treatments for type 1 diabetes, such as insulin. In one embodiment, the tolerogenic peptide of the present disclosure may be combined with one or more tolerogenic peptides derived from other proteins.

In one teaching, the pharmaceutical composition may be provided in the form of a kit for use according to the present disclosure, in which some or each of the peptide(s) is provided for simultaneous, separate or sequential administration. Alternatively, or in addition, if the pharmaceutical composition is to be administered in multiple doses, each dose may be packaged separately.

The induction of tolerance to GAD65 may be monitored in vivo by looking for a reduction in the level of GAD65 autoantibodies, CD4+ T-cells specific for GAD65 and/or B-cells capable of secreting GAD65 autoantibodies using techniques known in the art.

The induction of tolerance may be monitored by various techniques including the induction of anergy in CD4+ T-cells (which can be detected by subsequent challenge with antigen in vitro) and/or changes in the CD4+ T-cell population. The changes in the CD4+ T-cell population may include reduction in proliferation, downregulation in the production of IL-2, IFN-γ and/or IL-4, and increase in the production of IL-10.

In an alternative teaching, the tolerogenic peptides of the present disclosure may be in the form of a kit comprising one or more tolerogenic peptides as disclosed herein, or a modified tolerogenic peptide. For instance, these kits may comprise tolerogenic peptides for use in in vitro assays for generation of antigen-specific T-cells or detection of antigen-specific T-cells. In one embodiment, the peptide(s) may be further labelled with one or more moieties selected from: radionuclide, peptide tag, luminescent molecule, fluorescent molecule, quencher molecule, pH sensitive molecule, oxygen sensitive molecule or a combination thereof, for example.

### DETAILED DESCRIPTION

The present disclosure is further described by way of example and with reference to the figures, which show:

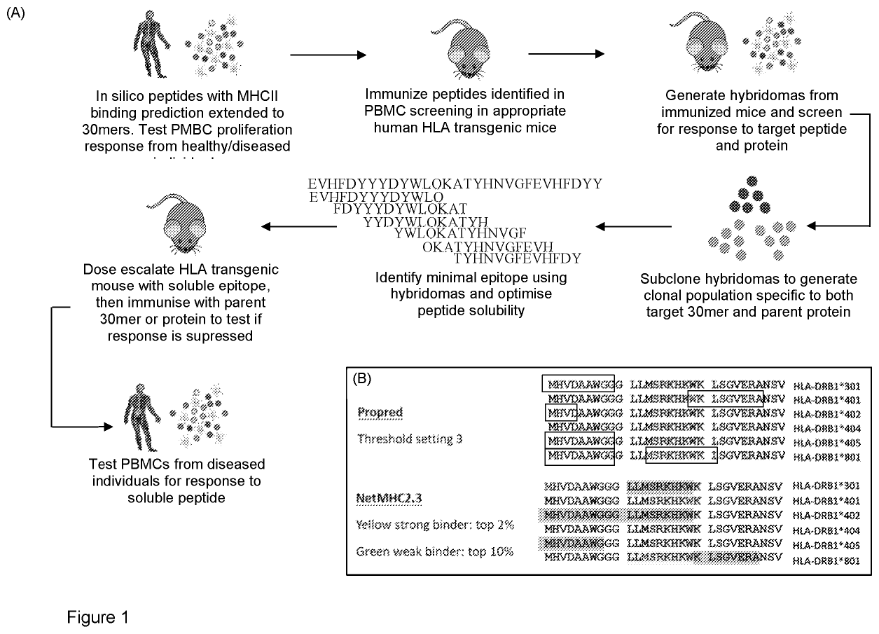

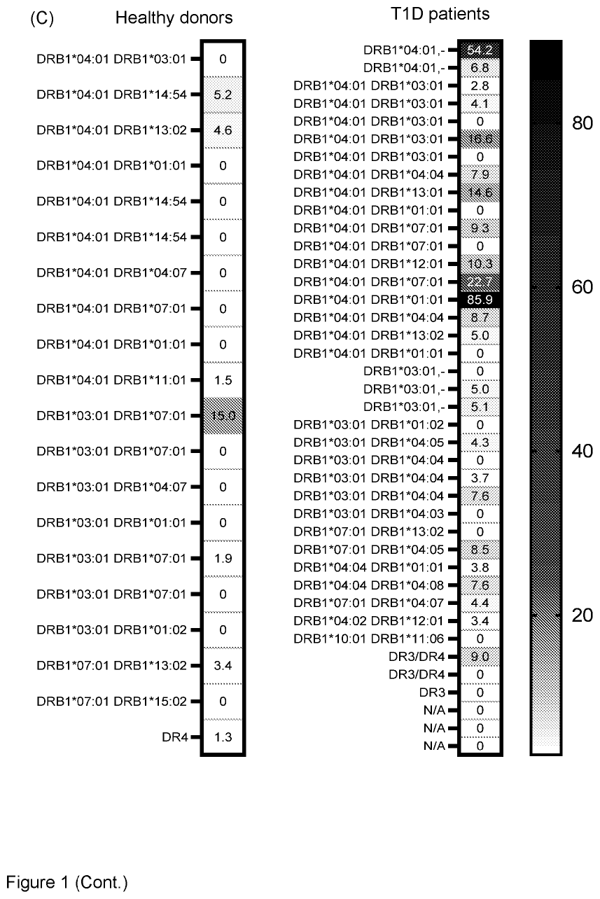

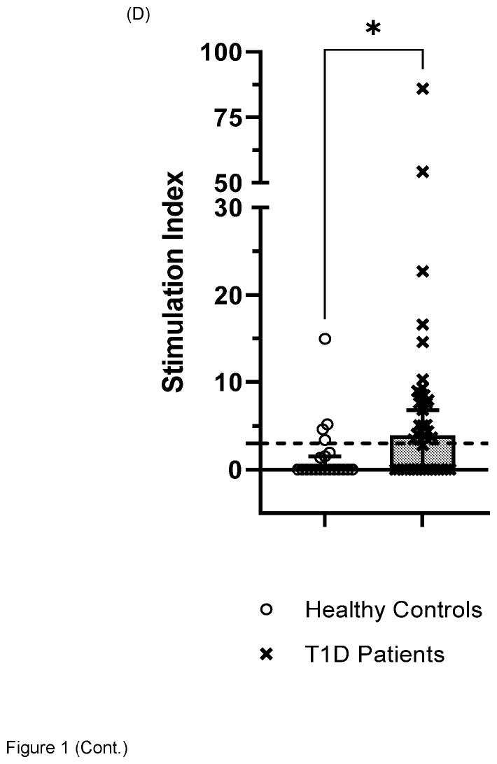

FIG. 1. Design and initial screening of P10 peptide from GAD65 protein. (A) Illustrates our strategy to identify tolerogenic peptide epitopes for treatment of patients with type I diabetes (T1D). (B) In silico MHC class II binding predictions for HLA-DR types associated with T1D within the P10 30-mer of GAD65. Text included in the boxes are predictions made by Propred; whilst highlighted text are predictions made by NetMHC2.3. (C) Day 7 stimulation index (SI) heatmaps for ³H-thymidine incorporation assay for peripheral blood mononuclear cells (PBMCs) stimulated with GAD65 P10 30-mer from non-diabetic healthy controls (left) vs T1D patients (right); the SI values are shown and positive responses have an SI≥3. Patient and healthy donor data are ordered by the presence of T1D associated DRB1 sub-alleles where mid-resolution genotype data was available, by HLA-DR serotype where only low-resolution typing was available or listed as N/A where low-resolution typing was inconclusive and insufficient sample for mid-resolution. DRB1*04:01,-indicates homozygosity for the allele. (D) Analysis of SI from day 7 of GAD65 P10 30-mer screen. Bar graphs show median and 95% confidence interval. Threshold SI of 3 indicated by dotted black line. Mann-Whitney test.

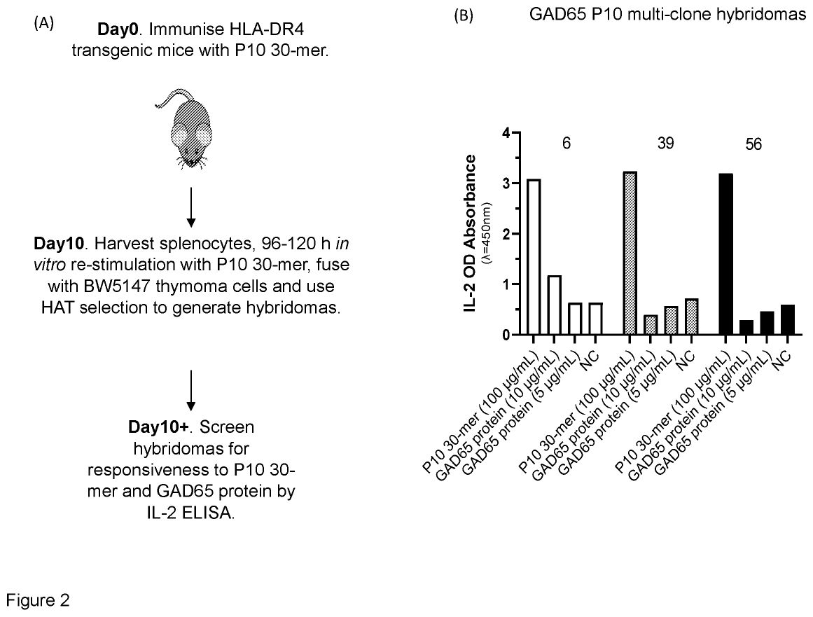

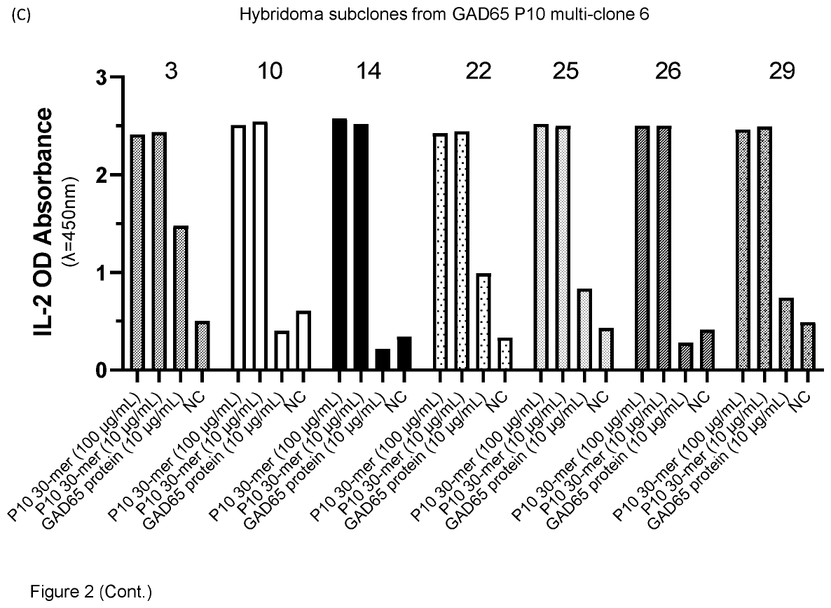

FIG. 2. GAD65 P10-specific hybridoma generation. (A) Illustrates the approach to generate T-cell hybridomas specific for GAD65 P10 30-mer. (B) Examples of GAD65 P10 specific multi-clone hybridomas producing IL-2 after co-culture with Preiss cells alone, or with either P10 30-mer or GAD65 protein. (C) Examples of IL-2 production from GAD65 P10 ssubclone hybridomas (subcloned from multi-clone 6) co-cultured for 48 h with Preiss cells alone, or with either P10 30-mer or GAD65 protein. NC; media only negative control.

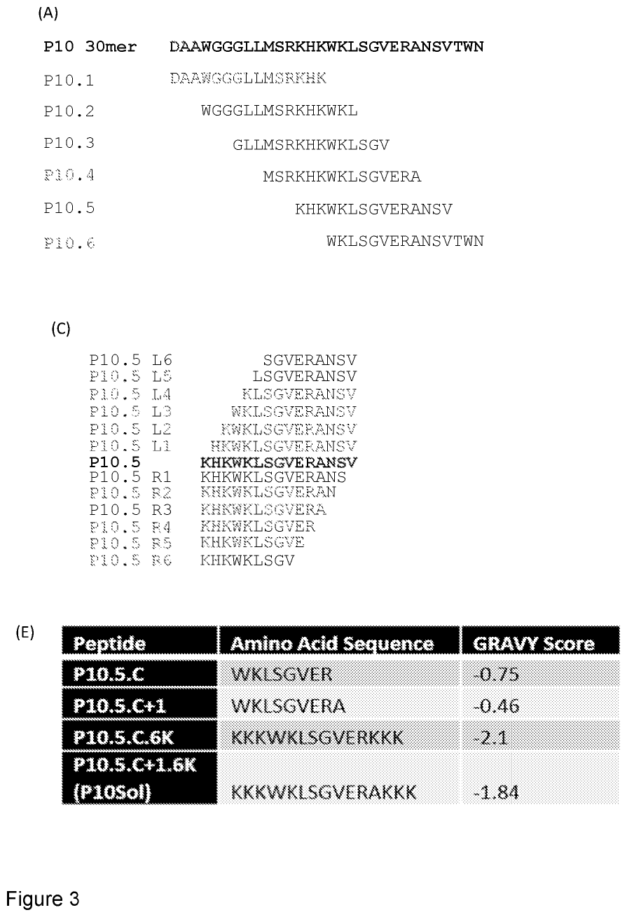

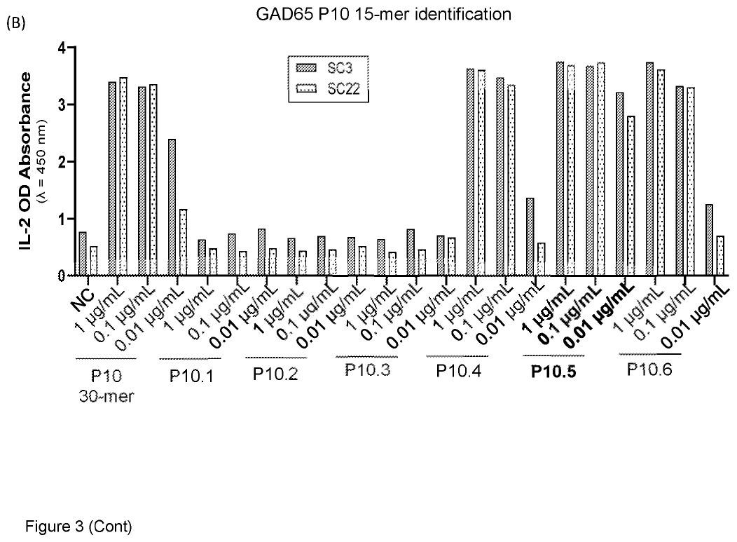

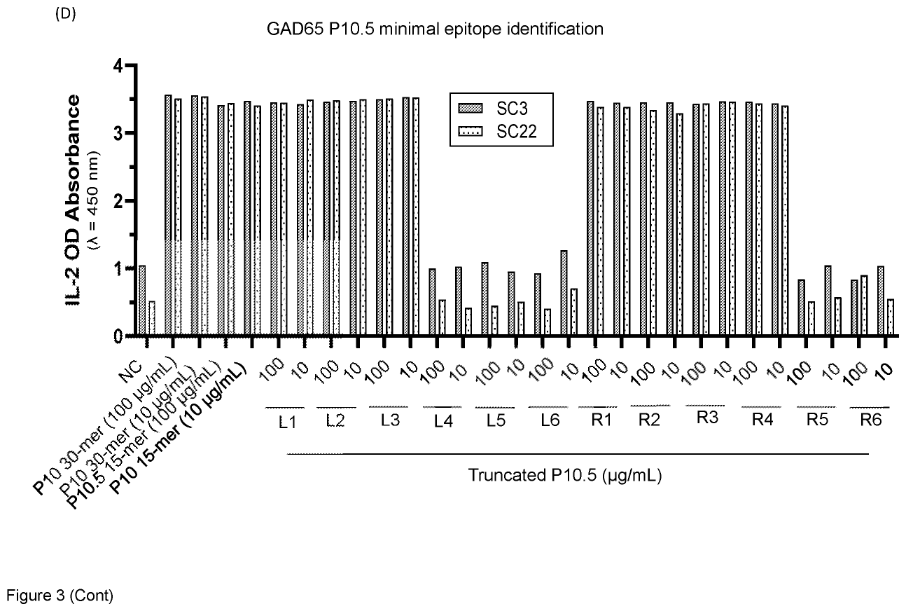

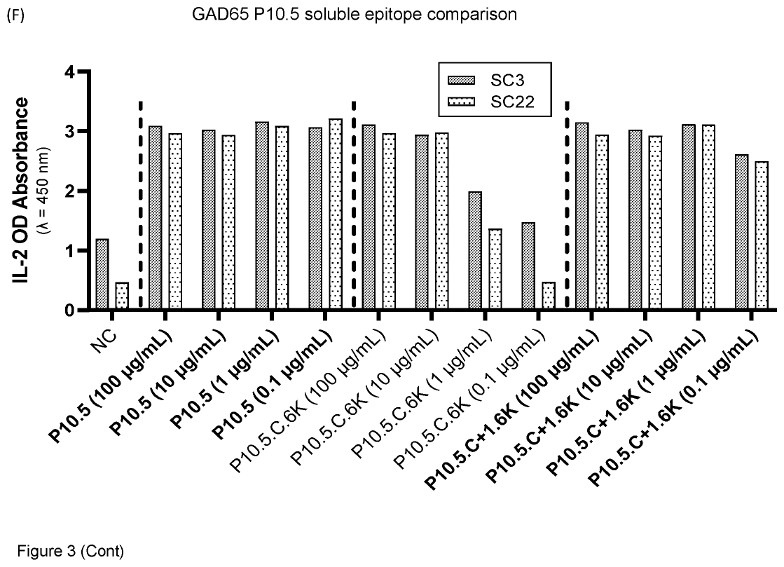

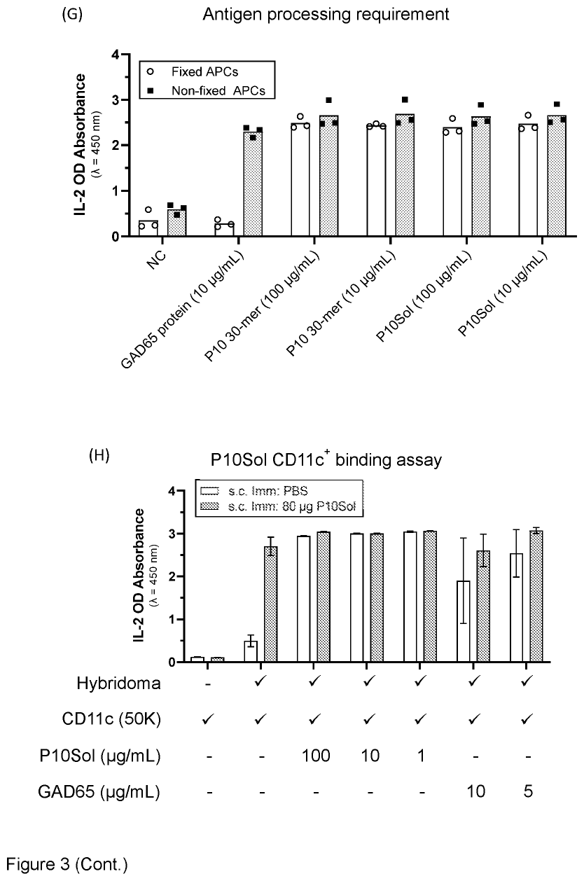

FIG. 3. Elucidation, design, and screening of a soluble minimal P10 epitope. (A) Illustration of 15-mer sequences shifting by 3 amino acids from N-terminus of the P10 30-mer. (B) Secreted IL-2 from 48 h co-culture of P10-specific single-clone hybridomas 3 and 22 (SC3 and SC22) with Preiss cells and 15-mer peptides. (C) Sequences of truncated peptides derived from the P10.5 15-mer. (D) Secreted IL-2 from 48 h co-culture of SC3 and SC22 with Preiss cells and truncated peptides. (E) Sequences and GRAVY hydrophobicity scores of the P10.5 core epitope and solubilised iterations. (F) IL-2 response from 48 h co-culture of SC3 and SC22 with Preiss cells and solubilised core epitopes derived from the P10.5 15-mer. (G) SC3 was co-cultured with either formaldehyde fixed or non-fixed HLA-DR4 transgenic mouse splenocytes as APCs, and stimulated with GAD65 protein, P10 30-mer or P10.5.C+1.6K (P10Sol; SEQ ID NO 23). Results are from n=2 independent experiments. (H) IL-2 response of SC3 and SC22 co-cultured for 48 h with CD11c⁺ splenocytes isolated from HLA-DR4 transgenic mice 1 h after subcutaneous injection with 80 μg of P10Sol or PBS and either no exogenous stimulant or P10Sol or GAD65 protein added in vitro.

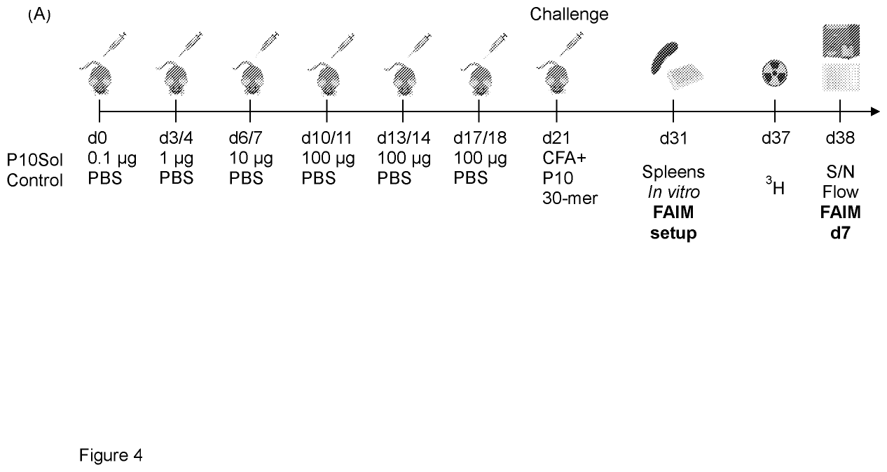

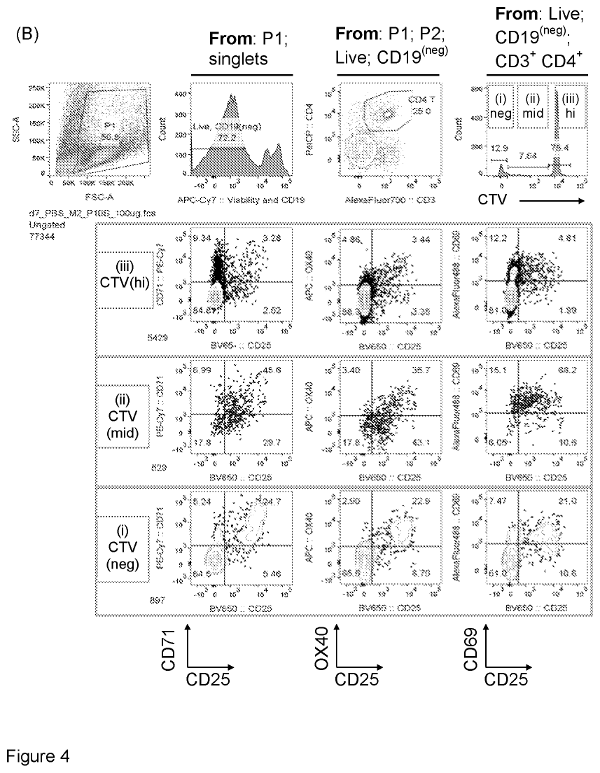

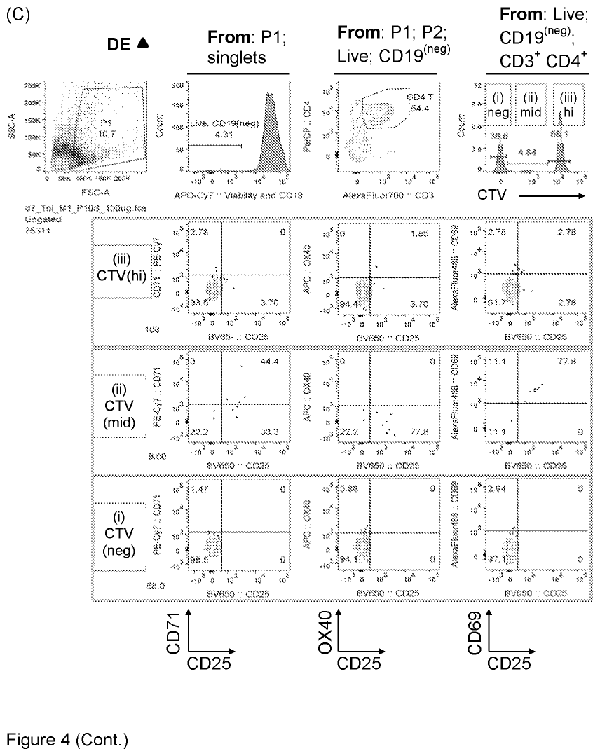

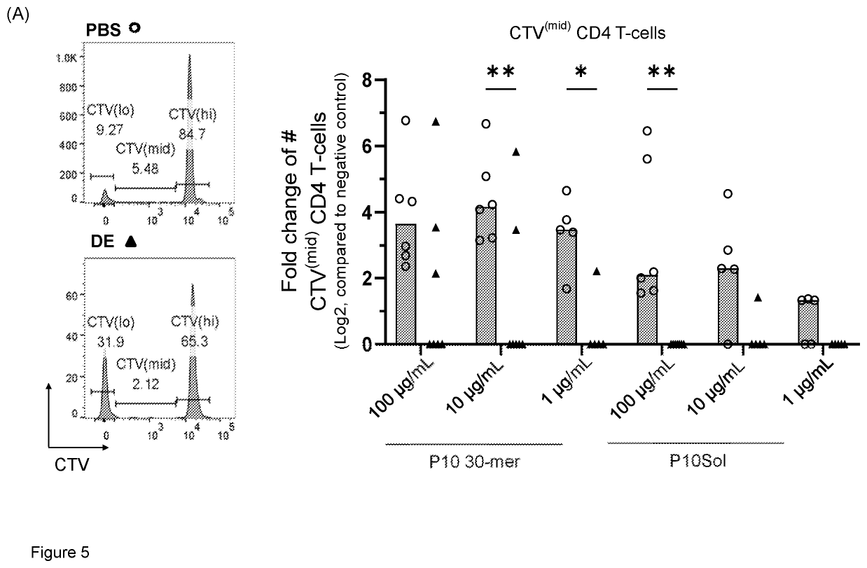

FIG. 4. Assessment of whether GAD65 P10Sol can induce immune tolerance. (A) Schematic to show mouse treatment schedule, challenge, Fluorescent dye dilution and Activation Induced Marker (FAIM) assay and readouts to test whether P10Sol can induce tolerance in HLA-DR4 transgenic mice, created with BioRender.com. Challenge; both GAD65 P10Sol dose escalation and PBS control groups challenged with 100 μg GAD65 P10 30-mer in Complete Freund's Adjuvant (CFA), spleens and in vitro; splenocytes isolated for CD4 selection, CTV labelling, recombination with unlabelled splenocytes and in vitro re-stimulation and culture, S/N; culture supernatants analysed for cytokine production, Flow; flow cytometric analysis. (B-C) Gating schematics to demonstrate how proliferating (CTV^{(mid)}) CD4 T-cells were analysed for activation marker expression. Positive expression gates for markers CD25, CD71, OX40 and CD69 were set using non-responding CTV labelled CD4 T-cells (CTV^{(hi)}) and applied to proliferated CTV^{(mid)} CD4 T-cells. (B) PBS treated mouse re-stimulated in vitro with 100 μg/mL GAD65 P10Sol. (C) GAD65 P10Sol dose escalation treated mouse re-stimulated in vitro with 100 μg/mL GAD65 P10Sol.

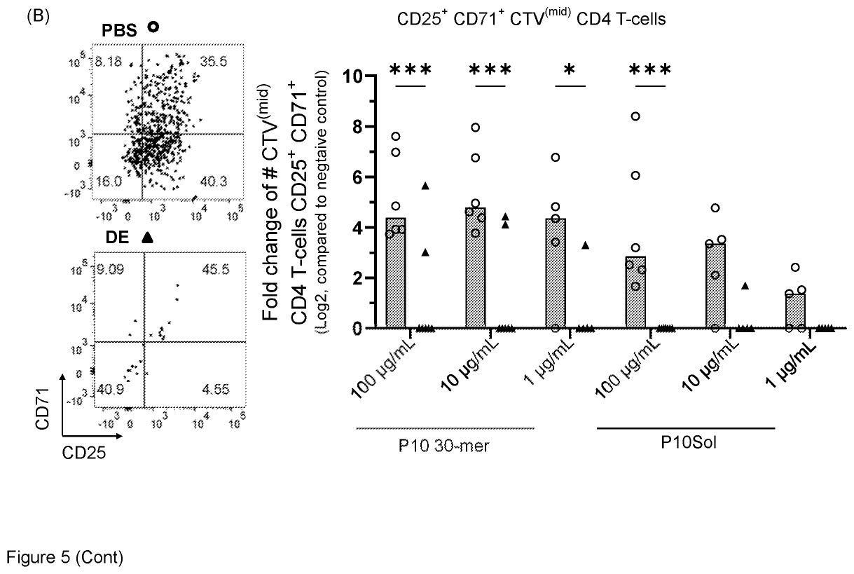

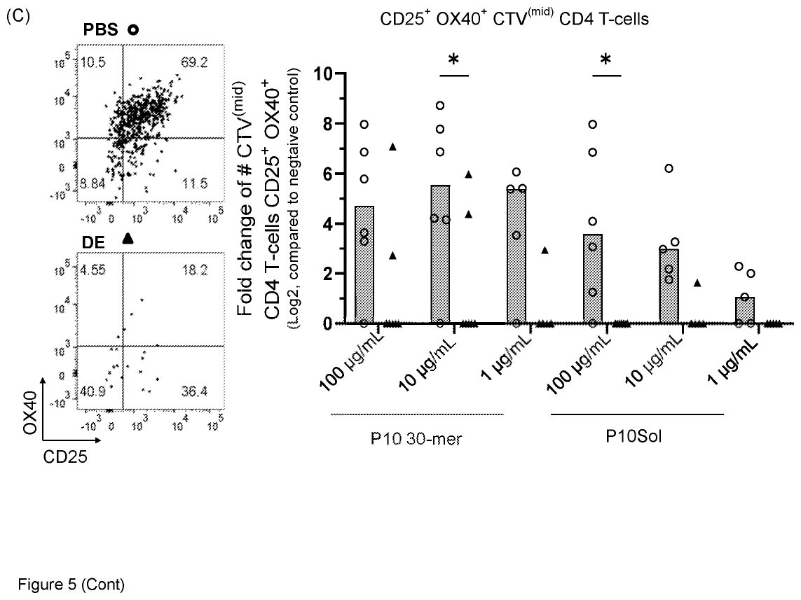

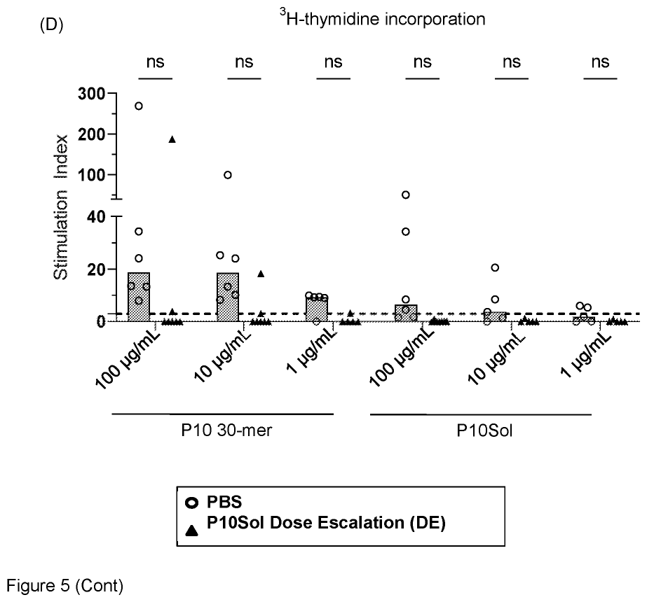

FIG. 5. GAD65 P10Sol can induce tolerance. Tolerance induction assessed by stimulation indices of flow cytometric parameters; proliferating CTV^{(mid)} CD4 T-cells (A), CD25⁺ CD71⁺ proliferating CD4 T-cells (B), CD25⁺ OX40⁺ proliferating CD4 T-cells (C) and by paired ³H-thymidine incorporation data (D). Representative flow cytometric plots are shown for cells from PBS or dose escalation treated mice re-stimulated and cultured with 10 μg/mL of P10 30-mer. For ³H-thymidine data, threshold stimulation index (SI) of 3 indicated by dashed black line. Statistical tests: Sidak's multiple comparisons. PBS treated mice n=6, P10Sol dose escalation treated mice n=7, results are from n=3 independent in vivo tolerance experiments.

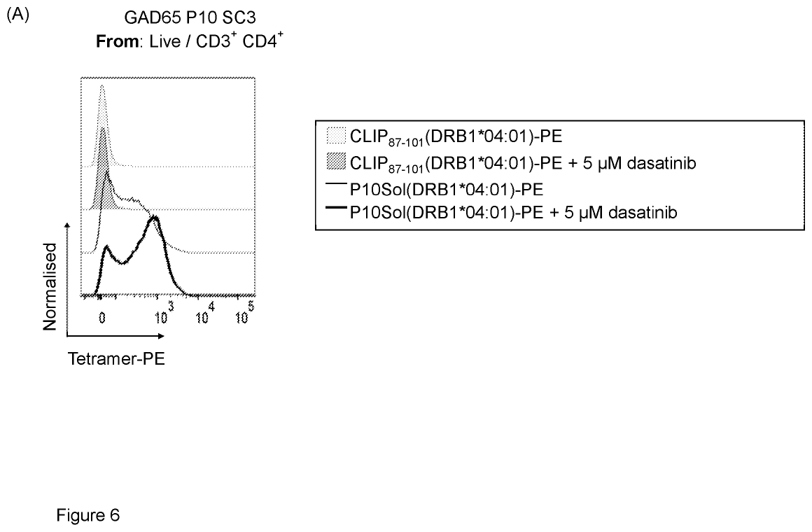

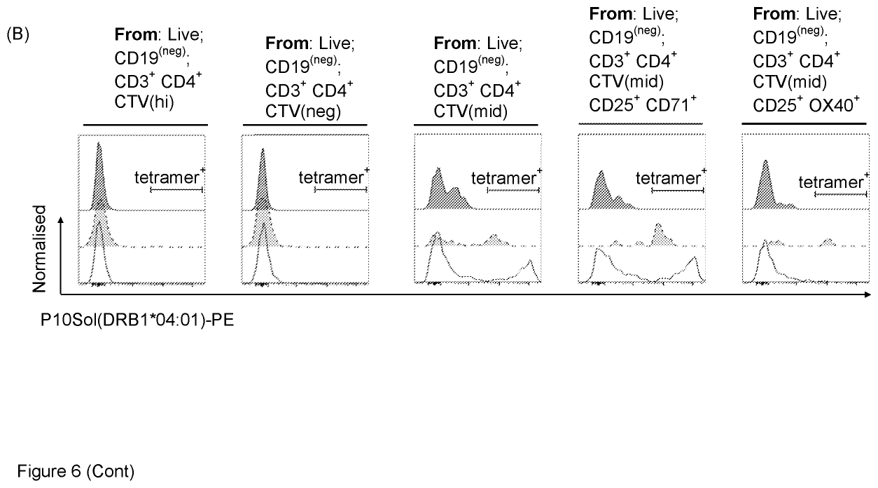

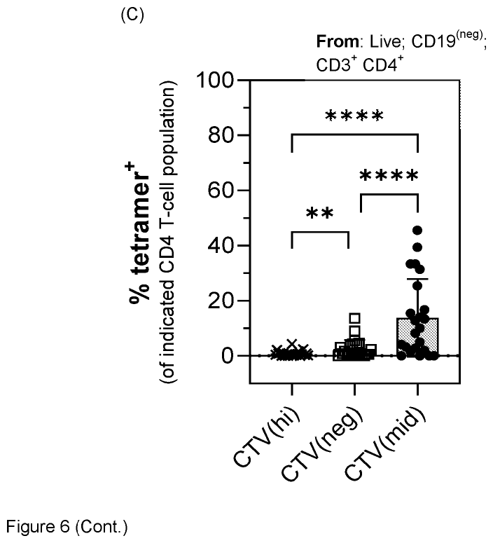

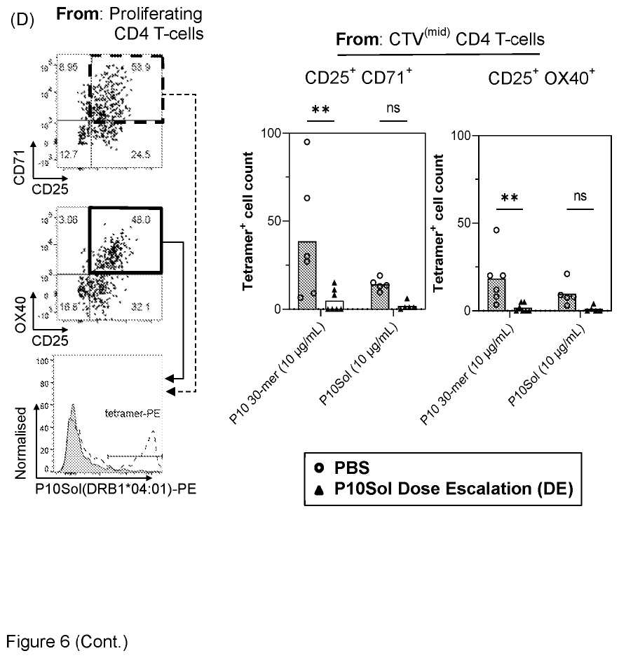

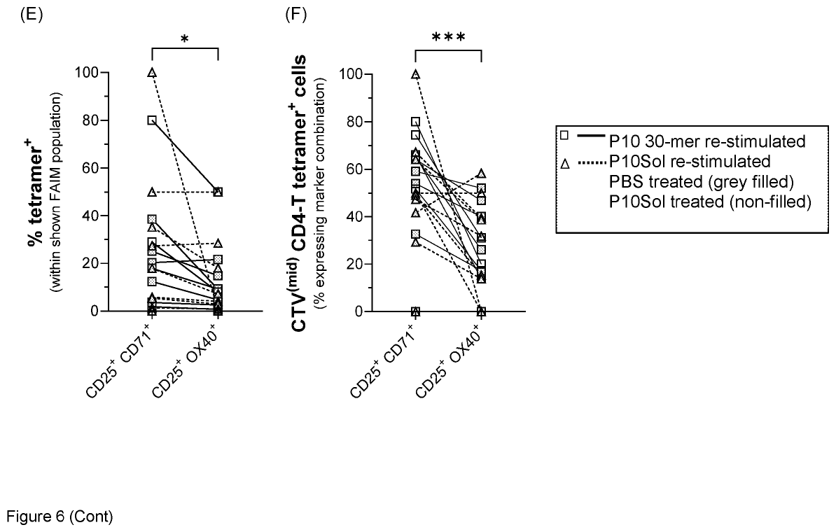

FIG. 6. GAD65 P10Sol MHC class II tetramer staining is enriched in CD71⁺ FAIM⁺ responding CD4 T-cells. (A) Validation of P10Sol (DRB1*04:01)-PE tetramer. GAD65 P10-specific SC3 hybridoma cells were stained at 37° C. for 1 h in the dark with either P10Sol (DRB1*04:01)-PE tetramer or control CLIP₈₇₋₁₀₁ (DRB1*04:01)-PE tetramer (both at 15 nM final concentration) in a stain mix which contained 5 UM dasatinib if the cells were pre-treated with dasatinib. This was followed by cell-surface anti-CD3, anti-CD4 and viability stain. Tetramer-PE overlays are from live, CD3⁺ CD4⁺ GAD65 P10 SC3 hybridoma cells. (B-F) P10Sol (DRB1*04:01)-PE tetramer staining in the FAIM assay used to test P10Sol tolerance induction described in FIGS. 4 and 5. (B) Example histogram overlays for P10Sol (DRB1*04:01)-PE tetramer staining in the indicated cell population. Fluorescence minus one (FMO) control are cells from a PBS treated mouse re-stimulated with 100 μg/mL GAD65 P10Sol and stained for cell-surface markers excluding the P10Sol (DRB1*04:01)-PE tetramer stain, black shaded trace (top). Cells from a PBS treated mouse re-stimulated in vitro with 10 μg/mL GAD65 P10 30-mer with full staining panel, solid line unfilled trace (bottom). Cells from a GAD65 P10Sol dose escalation treated mouse re-stimulated in vitro with 10 μg/mL GAD65 P10 30-mer with full staining panel, dashed line shaded trace (middle). (C) Tetramer binding within non-responsive (CTV^{(hi)}) CD4 T-cells, proliferating CD4 T-cells (CTV^{(mid)}), and CTV non-labelled CD4 T-cells (CTV^{(neg)}. (D) Example overlay comparison of P10Sol (DRB1*04:01)-PE tetramer staining in CD25⁺ CD71⁺ (dashed line unfilled trace) or CD25⁺ OX40⁺ (solid line shaded trace) FAIM⁺ populations from PBS treated mouse re-stimulated with 10 μg/mL P10 30-mer (D, left), with tetramer⁺ absolute numbers in FAIM⁺ cells separated by treatment type (D, right). PBS treated mice, open circles. P10Sol dose escalation treated mice, black filled triangles. Comparison of tetramer positivity rate within CTV^{(mid)} FAIM⁺ population (G) and the frequency of CTV^{(mid)} tetramer⁺ CD4 T-cells which express FAIM combinations (H). PBS treated mice n=6, P10Sol dose escalation treated mice n=7, from three independent experiments. Statistical tests: Tukey's multiple comparisons test (C), paired t-(G) and Wilcoxon matched pairs signed rank (H).

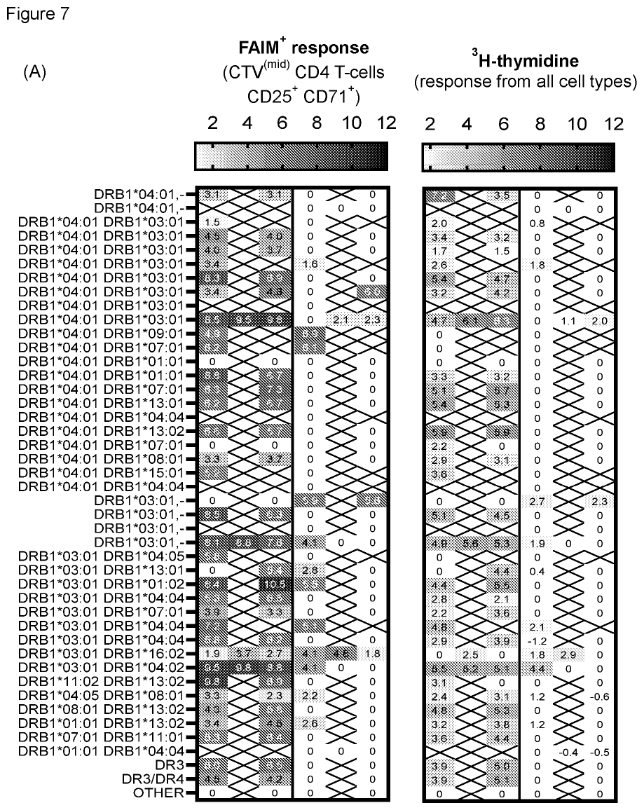

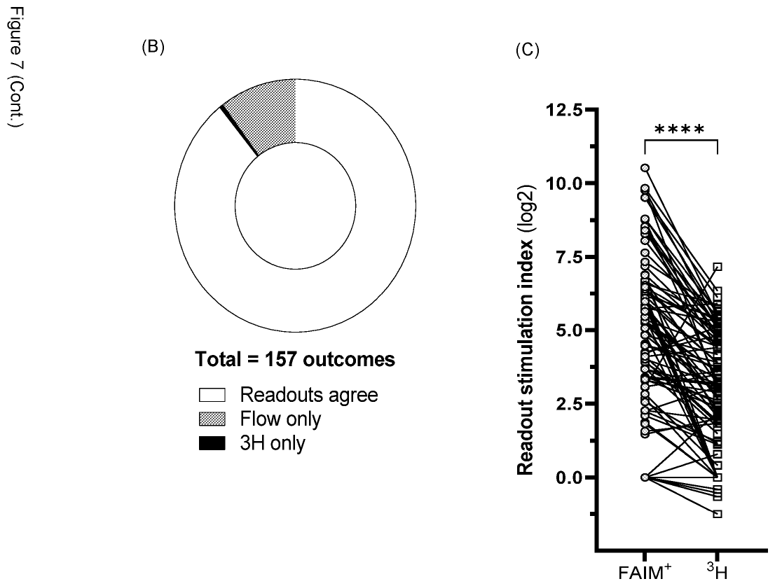

FIG. 7. GAD65 P10Sol response rate in T1D patients. (A) T1D PBMCs were screened using the FAIM assay for responsiveness to GAD65 P10 30-mer and GAD65 P10Sol with paired ³H-thymidine samples were acquired for each condition. A positive response for FAIM assay required a proliferating CD25⁺ CD71⁺ CD4 T-cell count >25 and the population SI to be ≥2× the negative control. A positive response for the ³H-thymidine assay required raw counts >1000 and a SI of ≥3× the negative control. White cells with the number zero indicate that this condition failed to induce a response which met the assay readout positive threshold, whereas a cell with a cross indicates that peptide concentration was not tested for that patient. The scale for both heat maps is Log 2 (stimulation index (SI)). Patient data is ordered by the presence of T1D associated DRB1 sub-alleles where mid-resolution genotype data was available or by HLA-DR serotype where only low-resolution typing was available. DRB1*04:01,-indicates homozygosity for the allele. (B) Agreement analysis for the outcomes of the flow cytometric and ³H-thymidine proliferation readouts of the FAIM assay. (C) Paired analysis of stimulation indexes from FAIM⁺ and ³H-thymidine readouts, wilcoxon matched-pairs sign rank test.

### Methods

### In Silico Prediction of Pan HLA Binding Peptides

MHC II binding predictions were conducted in silico using ProPred and NetMHCII-2.3 programmes (Singh and Raghava 2001, Jensen et al., 2018) to predict pan HLA-DRB1 binding 30-mer peptides for the GAD65 human protein.

### Peptide/Protein Antigens

Peptides were synthesized by GL Biochem (Shanghai) Ltd or Genscript (Leiden, The Netherlands). Peptides were >90% purity, resuspended from lyophilised powder in either 100% v/v dimethyl sulfoxide (DMSO) for 30-mers or PBS for soluble peptides. GAD65 protein was synthesised by Biologics Corporation. Purified protein derivative (PPD; Prionics; 7600060) was used at 300 IU/mL. Keyhole limpet haemocyanin (KLH; ThermoFisher Scientific; 77600) was used at 20 μg/mL.

### Human PBMC Isolation and ³H-Thymidine Incorporation Assay

Fresh blood samples were collected in CPDA-tubes. PBMCs from lymphocyte cones or fresh blood were isolated by Ficoll gradient centrifugation, frozen in 40% RPMI-1640, 50% heat inactivated fetal bovine serum (Sigma; F9665) and 10% DMSO and stored in liquid nitrogen until required. Thawed PBMCs were cultured at 1.5×10⁶ cells/mL in X-VIVO-15 medium ((Lonza BE02-061Q) supplemented with 5% v/v human AB serum (Sigma H4522), 1× penicillin/streptomycin (Gibco 15140122)) either in the presence of GAD65 P10 30-mer antigen between 20-50 μg/mL or absence of antigen (negative control). Peptide response was measured by 3H radioactive thymidine incorporation on day 5 and day 7, by pulsing cell cultures with ³H-thymidine (Perkin Elmer), as previously described (Mazza et al., 2002). Positive responses had corrected counts per minute (ccpm) counts >1000 and a stimulation index (SI)≥3, calculated as the fold-change of peptide stimulated condition over the negative control.

### HLA-DR Typing

Genomic DNA was extracted from 1-5×10⁶ PBMCs (Qiagen; 69504). Low-resolution HLA-DR serotype was interpreted from the positive lanes after PCR analysis using the reagents and results tables from the HLA-DR Low typing kit (Olerup; 101.101-12u). Mid resolution HLA-DRB1 genotyping was provided by VH Bio.

### Mice

HLA-DR4 transgenic mice express HLA-DRA*0101, -DRB1*0401 and human CD4 are previously described (Fugger et al., 1994). B-cells from the peripheral blood of HLA-DR4 mice were phenotyped for HLA (clone TU39) and mouse MHCII (clone M5/114.15.2) by flow cytometry. Male and female mice aged between 6-12 weeks were used. Animals were housed under specific pathogen-free conditions in the Biomedical Services Unit of the University of Birmingham. Experiments were performed in accordance with the local ethical review panel and UK Home Office regulations.

### Generation and Screening of T-Cell Hybridomas

HLA-DR4 transgenic mice were injected subcutaneously with Complete Freund's Adjuvant (CFA) and 100 μg of GAD65 P10 30-mer. After 10 days, splenocytes were isolated and re-stimulated with GAD65 P10 30-mer for a further 4-5 days before fusion with hypoxanthine-aminopterin-thymidine (HAT) sensitive BW5147 cells using polyethylene glycol (PEG). Fused hybridomas were expanded in HAT selection media before antigen-specific screening. Co-cultures of 1×10⁵ hybridomas and 2×10⁵ Priess cells (an Epstein-Barr Virus (EBV) transformed DR4 (DRB1*04:01) expressing human cell line (ECACC 86052111)) as antigen presenting cells (APCs) were incubated for 48 h in a 96-well plate with peptide, whole GAD65 protein, or media only. Antigen-specific responses were determined by IL-2 secretion into culture supernatant by ELISA assay (BioLegend 431004). Hybridomas that proliferated in response to both GAD65 protein and GAD65 30-mer were single cell cloned by limiting dilution.

### Peptides

Six 15-mers that spanned the parent 30-mer amino acid sequence were generated by 3 amino acid shifts from the N-terminus toward the C-terminus. Twelve truncated peptides were generated by removing 1 amino acid for each peptide from the N- and C-termini of a hybridoma responsive 15-mer peptide, up to a maximum of six amino acids in either direction.

The hydrophilic amino acid lysine (K) was added to the N- and C-termini of the core epitope to create a more soluble peptide as determined by the GRAVY score (http://www.gravy-calculator.de/).

### Fixed APC T-Cell Hybridoma Screen

Splenocytes from HLA-DR4 transgenic mice were formaldehyde fixed by the following protocol performed at room temperature; 5 min incubation with 0.5% w/v formaldehyde in PBS at 1×10⁶ cells/mL quenched by addition of an equal volume of 0.4 M glycine in PBS solution for a further 5 min, before three washes with cold PBS. T-cell hybridoma screens were set up using 1×10⁵ hybridomas and either 2×10⁵ formaldehyde fixed APCs or 2×10⁵ non-fixed APCs for 48 h co-culture with stimulant or control and responses measured by IL-2 secretion.

### Steady State CD11c⁺ Peptide Binding Assay

HLA-DR4 transgenic mice were injected subcutaneously with 80 μg of GAD65 P10.5.C+1.6K (GAD65 P10Sol) peptide, before isolation of CD11c⁺ splenocytes 1 h later CD11c⁺ positive selection kit (Miltenyi; 130-125-835). T-cell hybridoma responses were measured by secreted IL-2 after a 48 h co-culture of 0.5×10⁵ CD11c⁺ cells with 1×10⁵ GAD65 P10-specific hybridomas with or without exogenous antigen added in vitro.

### Tolerance Induction and Immunisation Challenge

HLA-DR4 transgenic mice were injected subcutaneously with the GAD65 P10Sol peptide every 3 to 4 days with a dose escalation course of 0.1 μg, 1 μg, 10 μg, 100 μg, 100 μg, 100 μg. On day 21, all mice were challenged with 100 μg of the GAD65 P10 30-mer in CFA. Splenocytes were isolated 10 days after challenge and used in the FAIM assay.

### Examples

### Identification of Tolerogenic Antigen Processing Independent T-Cell Epitopes (Apitopes)

The workflow to design and validate tolerogenic apitopes (antigen processing independent T-cell epitopes) begins with identification of peptides predicted to bind to various HLA-DR molecules (pan-DR binders) using a combination of publicly available MHC-binding algorithms (FIG. 1A). In silico analyses identified 9 or 15 amino acid peptides within GAD65 that were extended to ˜30-mer peptides (FIG. 1B) to encourage antigen processing and presentation of a naturally processed epitope (Anderton et al., 2002). To test whether the designed 30-mer peptides could be correctly processed and preferentially induce immune responses in T1D patients, we screened for GAD65 30-mers responses in isolated peripheral blood mononuclear cells (PBMCs) from 20 non-diabetic healthy controls and 40 adults with T1D. HLA-DRB1*04:01 and/or HLA-DRB1*03:01 alleles are strongly associated with T1D disease (Pociot and McDermott 2002, Noble and Valdes 2011) and 45% (18/40) of the T1D patients tested possessed at least one HLA-DRB1*04:01 allele and 35% (14/40) had at least one HLA-DRB1*03:01 allele (FIG. 1C). To provide the most appropriate HLA comparators in the initial peptide screen, healthy control PBMCs were HLA-genotyped to ensure that a high proportion of the health cohort possessed HLA-DRB1*04:01 and/or HLA-DRB1*03:01 alleles (FIG. 1C). On day 7 of the ³H-thymidine incorporation assay, 20% (4/20) of healthy controls had shown a positive response to GAD65 P10 30-mer compared to 58% (23/40) of T1D patients (FIG. 1C). This demonstrated a significantly higher response rate in T1D patients (Fisher's exact test; p=0.0069). Furthermore, the average magnitude of the proliferative response was also significantly increased inby T1D patients (FIG. 1D; Mann-Whitney test p=0.0146).

Having demonstrated an increased responsiveness to the P10 30-mer peptide of GAD65 by T1D patients, the next steps, as illustrated in FIG. 1A, were to assess peptide immunogenicity in appropriate human HLA-DR transgenic mice and to use responsive HLA-DR transgenic mice to generate peptide specific hybridomas. HLA-DR4 transgenic mice, which express the HLA-DRB1*04:01 allele, responded well to GAD65 P10 30-mer immunisation (data not shown) and were used to generate P10 30-mer specific T-cell hybridomas (FIG. 2A). Generation of hybridomas that respond to both the GAD65 protein and the P10 30-mer peptide means that these hybridomas can recognise naturally processed peptide presented by APCs—a property shown to be critical for successful peptide immunotherapy design (Anderton et al 2002). Primary screens of hybridoma cultures with HLA-DRB1*04:01 expressing APCs indicated that hybridoma multi-clone 6 could respond to both P10 30-mer and whole GAD65 protein (FIG. 2B), with this multi-clone then being sub-cloned via limiting dilution to single cells (FIG. 2C).

### Enhancing Solubility of Tolerogenic Peptides

Peptide solubility is a key property required for tolerogenic apitope design (Shepard et al., 2021), so the inventors next sought to elucidate the P10 30-mer minimal core epitope and to test analogues modified to optimise solubility. This is achieved by identification of the responsive 15-mer within the 30-mer followed by sequential removal of amino acids from the N- and C-termini of the 15-mer to identify the minimal “core” amino acids critical for MHC class II binding and hybridoma TCR stimulation (FIG. 3A-D). Applying this methodology the inventors identified P10.5 15-mer within the P10 30-mer (FIG. 3B) and the minimal core epitope (P10.5.C; SEQ ID NO 20, FIG. 3D-E). Iterations designed around the core minimal epitope included retention of non-critical residues and/or addition of multiple lysine residues to both the N- and C-termini (FIG. 3E). Iterations P10.5.C.6K (SEQ ID NO 22) and P10.5.C+1.6K (SEQ ID NO 23) both had improved GRAVY scores of −2.1 and −1.84, respectively, and both peptides were highly soluble in PBS. Although P10.5.C.6K contains only the critical core epitope with additional lysine residues, the re-inclusion of the single non-critical alanine at the C-terminus (P10.5.C+1.6K), increased the potency of the single-clone hybridoma response by 10-fold (FIG. 3F).

### Binding of Tolerogenic Peptides to MHC Class II and APCs

The next step was to test if GAD65 P10.5.C+1.6K (P10Sol; SEQ ID NO 23) could bind to: (i) MHC class II molecules without antigen processing and, (ii) to steady-state CD11c⁺ APCs. These properties are good indicators of whether a peptide can induce tolerance and be characterised as an apitope (Shepard et al., 2021). Formaldehyde fixation of splenocytes inhibits antigen processing and so to test whether GAD65 P10Sol could bind directly to cell-surface MHC class II without antigen processing, GAD65 P10-specific hybridoma SC3 was co-cultured with fixed or non-fixed splenocytes from HLA-DR4 transgenic mice. Response to whole GAD65 protein required antigen processing from non-fixed splenocytes, whereas both GAD65 P10 30-mer and GAD65 P10Sol (SEQ ID NO 23) induced strong IL-2 responses from hybridoma SC3 when co-cultured with both fixed and non-fixed splenocytes (FIG. 3G). This demonstrated that both peptides could bind directly to MHC class II independent from antigen processing. To test whether P10Sol could bind to steady-state CD11c⁺ APCs in vivo, HLA-DR4 transgenic mice were injected with 80 μg of P10Sol or PBS, with CD11c⁺ splenocytes isolated 1 h later and co-cultured with GAD65 P10 hybridomas SC3 or SC22. Isolated CD11c⁺ cells from mice injected with P10Sol peptide could activate and induce strong IL-2 responses in P10-specific hybridomas without exogenous addition of peptide/protein to the in vitro culture, showing rapid and effective in vivo presentation of the soluble P10Sol peptide (FIG. 3H). However, for CD11c⁺ cells from PBS injected mice, P10-specific hybridoma IL-2 responses were only observed when P10Sol peptide or GAD65 protein were added exogenously to the in vitro culture (FIG. 3H). Taken together, this demonstrated that P10Sol can bind to steady-state CD11c⁺ APCs in an antigen processing independent manner, a characteristic of an apitope, and was thus a suitable candidate for tolerance induction studies.

### Tolerance Induction

Soluble candidate peptide GAD65 P10Sol (SEQ ID NO 23) was used as a representative peptide and tested for induction of tolerance in a suitable HLA-DR transgenic mouse model (FIG. 1A). HLA-DR4 transgenic mice were treated with either a dose escalation of GAD65 P10Sol or PBS control over 17/18 days before challenge with the P10 30-mer in strong adjuvant (FIG. 4A). On day 10 after challenge, spleens were harvested and the 7-day in vitro Fluorescent dye dilution Activation Induced Marker (FAIM) assay was set up (FIG. 4A) with in vitro re-stimulation using titrations of P10 30-mer and GAD65 P10Sol. The FAIM assay involves a fluorescent dye-labelled CD4⁺ cell enriched culture with a primary flow cytometric readout assessing CD4 T-cell fluorescent dye dilution and cell-surface activation marker expression with optional paired readouts of ³H-thymidine incorporation and secreted cytokine measurement. The FAIM assay was validated here by running parallel ³H-thymidine incorporation assays. In-house testing of the FAIM assay to detect induction of CD4 T-cell tolerance with B10PL mice and dose escalation of a tolerogenic peptide derived from Myelin Basic Protein (Burton et al., 2014), showed that cytokine, ³H-thymidine and flow cytometric readouts of the FAIM assay, including the use of CD71 as a new activation induced marker, could successfully distinguish tolerance induction (data not shown). This new activation induced marker, CD71, is a transferrin receptor involved in iron uptake, has expression patterns strongly associated with Ki67 and cell proliferation (Lašt'ovička et al., 2009; Motamedi et al., 2016). Flow cytometric analysis (gating strategy for a PBS treated and a P10Sol treated mouse shown in FIG. 4B-C) from day 7 of the in vitro re-stimulation which used titrations of GAD65 P10 30-mer and P10Sol revealed that total CTV diluted proliferating (CTV^{(mid)}) CD4 T-cells were significantly reduced in P10Sol treated mice (FIG. 5A; Sidak's multiple comparisons test). Furthermore, in P10Sol treated mice, antigen-responding CTV^{(mid)} CD4 T-cells which were co-expressing combinations of activation markers CD25/CD71 or CD25/OX40, were either almost absent or significantly reduced (FIG. 5B-C; Sidák's multiple comparisons test). This reduced response measured by the flow cytometric readout of the FAIM assay was supported by the paired ³H-thymidine incorporation readout, which showed a strong but not statistically significant trend towards reduced overall proliferative response in cultured cells from P10Sol treated mice re-stimulated in vitro with P10Sol or P10 30-mer (FIG. 5D). We tetramerised and validated a P10Sol peptide-DRB1*04:01 MHCII monomer, P10Sol (DRB1*04:01)-PE (FIG. 6A) and used this to assess whether proliferating CD4 T-cells which expressed activation markers were antigen-specific. Tetramer stain was included in samples stimulated with 10 μg/mL of P10Sol or P10 30-mer and showed selective binding to antigen-responsive proliferating CD4 T-cells (FIG. 6B-C). The number of antigen-specific tetramer⁺ FAIM⁺ cells was highly enriched in PBS treated mice and almost absent in mice treated with P10Sol dose escalation (FIG. 6D). Furthermore, the combination of CD25/CD71 on responding CD4 T-cells identified a significantly increased frequency of tetramer⁺ cells than CD25/OX40 (FIG. 6E). This also held true when enrichment of activation marker co-expression within the total tetramer⁺ CD4 T-cells was analysed (FIG. 6F). P10Sol can directly bind MHCII without antigen processing and has demonstrated the ability to induce tolerance in HLA-DR4 transgenic mice.

Having demonstrated the properties of and responses to P10Sol in T-cell hybridomas and HLA-DR4 transgenic mice, it was next critical to assess whether T1D patients could also respond to P10Sol. We used the FAIM assay with PBMCs from a cohort of 44 adult T1D patients to measure paired FAIM⁺ (CD25⁺ CD71⁺ CTV^{(mid)} CD4⁺ T-cell) and ³H-thymidine proliferation responses (FIG. 7A). FAIM⁺ analysis highlighted that 89% (34/38) of T1D patients could respond to GAD65 P10 30-mer, of which 38% (13/34) also responded to P10Sol (FIG. 7A). Importantly, the FAIM⁺ flow cytometric readout detected 16 additional positive responses (10.2% of total outcomes) which were below the positive threshold of the paired ³H-thymidine proliferation readout, whereas only 1 outcome (0.6% of total outcomes) was detected by ³H-thymidine and not FAIM⁺ (FIG. 7A-B). This demonstrated that P10Sol is a natural epitope recognised by TCRs of individuals with type 1 diabetes.

### CONCLUSION

We have used a combination of ³H-thymidine incorporation and activation induced marker-based methods to identify and validate novel peptide epitopes capable of inducing immune tolerance towards the self-antigen. Using P10Sol as a representative peptide, dose escalation was able to inhibit CD4⁺ T-cell specific proliferation measured by flow cytometry, which was corroborated by the paired ³H-thymidine incorporation readout. In addition, the number of CD4⁺ T-cells that were proliferative and co-expressed activation markers CD25⁺ OX40⁺ and CD25⁺ CD71⁺ were reduced in mice treated with P10Sol dose escalation. Using a P10Sol peptide-MHC class II tetramer, a higher frequency of antigen-specific proliferating CD4 T-cells were identified using only the new combination of activation markers CD25 and CD71, compared to the use of only CD25 and OX40, which thus validated the new combination of CD25 and CD71. We have then demonstrated that P10Sol, identified by the described methods, can induce CD4 T-cell responses in the PBMCs of patients with T1D, is a disease relevant epitope and warrants further clinical development.

### REFERENCES

Anderton, S., Viner, N., Matharu, P. et al. Influence of a dominant cryptic epitope on autoimmune T cell tolerance. Nat Immunol 3, 175-181 (2002). https://doi.org/10.1038/ni756
Atkinson et al., 2014. Lancet. 2014 Jan. 4; 383 (9911): 69-82. doi: 10.1016/S0140-6736 (13) 60591-7.
Babon et al., 2016. Nat Med 2016 December; 22 (12): 1482-1487. doi: 10.1038/nm.4203. Epub 2016 Oct. 31
Burton et al., 2014. Nat Commun. 2014 Sep. 3; 5:4741. doi: 10.1038/ncomms5741
Feutren et al., 1986. Lancet. 1986 Jul. 19; 2 (8499): 119-24. doi: 10.1016/s0140-6736 (86) 91943-4.
Fugger et al., 1994. Proc Natl Acad Sci USA. 1994 Jun. 21; 91 (13): 6151-5. doi: 10.1073/pnas.91.13.6151
Herold et al., 2019. N Engl J Med. 2019 Aug. 15; 381 (7): 603-613. doi: 10.1056/NEJMoa1902226.
Jensen et al., 2018. Immunology. 2018 July; 154 (3): 394-406. Doi: 10.1111/imm.12889. Epub 2018 Feb. 6
Kroll et al., 2013. J Clin Virol. 2013 June; 57 (2): 115-119.
Lašt'ovička et al., 2009. Cell Immunol. 2009; 256 (1-2): 79-85. doi: 10.1016/j.cellimm.2009.01.007
Mazza et al., 2002. Clin Exp Immunol. 2002 June; 128 (3): 538-47. doi: 10.1046/j. 1365-2249.2002.01831.x
Motamedi et al., 2016. J Immunol Methods. 2016 doi: 10.1016/j.jim.2016.08.002 Oct; 437:43-52.
Mullard 2022. Nature Reviews Drug Discovery. Doi 10.1038/D41573-022-00198-9
Naruse et al., 1997. Tissue Antigens. 1997 February; 49 (2): 152-9. doi: 10.1111/j. 1399-0039.1997.tb02729.x
Noble and Valdes 2011. Curr Diab Rep. 2011 December; 11 (6): 533-42. doi: 10.1007/s11892-011-0223-x
Pociot and McDermott 2002. Genes Immun. 2002 August; 3 (5): 235-49. Doi: 10.1038/sj.gene.6363875.
Peterson and Haskins 1996. Diabetes. Mar; 45 (3): 328-36. doi: 10.2337/diab.45.3.328
Shepard et al., 2021. Front Immunol. 2021 Apr. 14; 12:654201. doi: 10.3389/fimmu.2021.654201
Singh and Raghava 2001. Bioinformatics. 2001 December; 17 (12): 1236-7. Doi: 10.1093/bioinformatics/17.12.1236
Wraith, D. Antigen-specific immunotherapy. Nature 530, 422-423 (2016). https://doi.org/10.1038/nature 17300

1. A method of treating type-1 diabetes, the method comprising administering to a subject in need thereof a tolerogenic peptide capable of binding an MHC class II molecule independent of antigen processing for use in the treatment of type 1 diabetes, wherein the peptide comprises a fragment of glutamate decarboxylase 65 (GAD65) or a variant thereof.

2. (canceled)

3. The method according to claim 1, wherein the fragment of GAD65 comprises 5 to 40, 5 to 20, or 5 to 15 amino acids in length.

4. (canceled)

5. The method according to claim 1, wherein the peptide is selected from SEQ ID NO: 20 to 23.

6. (canceled)

7. The method according to claim 1, wherein the peptide comprises the amino acid sequence DAAWGGGLLMSRKHKWKLSGVERANSVTWN, IFSPGGAISNMYAMMIARFKMFPEVKEKGMA, DLERRILEAKQKGFVPFLVSATAGTTVYGA, or fragment thereof.

8.-9. (canceled)

10. The method according to claim 7, wherein the peptide comprises one or more amino acid modifications selected from:
(i) one or more amino acid substitutions (including conservative substitutions),
(ii) one or more amino acid deletions,
(iii) one or more amino acid additions and/or
(iv) one or more sequence inversions.

11. The method according to claim 10, wherein the peptide is selected from SEQ ID NO: 1 to 65, or SEQ ID NO:1 to 23.

12. (canceled)

13. The tolerogenic peptide for use according to claim 1, wherein the peptide further comprises one or more amino acid modifications that increase the solubility of the peptide.

14. The tolerogenic peptide for use according to claim 13, wherein the one or more amino acid modifications comprise addition and/or substitution of a wild-type or reference residue with one or more amino acids selected from lysine, arginine, histidine, aspartic acid, glutamic acid, serine, threonine, asparagine and/or glutamine.

15. The tolerogenic peptide for use according to claim 13, wherein the one or more amino acid modifications comprise addition of one to four lysine residues to the N-terminus and/or C-terminus of the peptide.

16. The tolerogenic peptide for use according to claim 15, wherein the peptide is selected from KKKWKLSGVERKKK, KKKWKLSGVERAKKK, KKKRISNMYAMMIARRKKK, KKKKISNMYAMMIARKKKK and/or KKERRILEAKQKGFVPKK.

17. (canceled)

18. A pharmaceutical composition comprising an effective amount of one or more tolerogenic peptides selected from SEQ ID NO: 1 to 65; and optionally, wherein the peptide further comprises one or more amino acid modifications that increase the solubility of the peptide.

19.-20. (canceled)

21. A tolerogenic peptide capable of binding an MHC class II molecule independent of antigen processing, wherein the peptide derived from a pancreatic cell comprises a fragment of glutamate decarboxylase 65 (GAD65) and comprises the sequence WKLSGVER, ISNMYAMMIA or ERRILEAKOKGFVP.

22.-29. (canceled)

30. The tolerogenic peptide according to claim 21, wherein the peptide is selected from SEQ ID NO: 1 to 65.

31. The tolerogenic peptide according to claim 30, wherein the peptide further comprises one or more amino acid modifications that increase the solubility of the peptide.

32. The tolerogenic peptide according to claim 31, wherein the one or more amino acid modifications comprise addition and/or substitution of a wild-type or reference residue with one or more amino acids selected from lysine, arginine, histidine, aspartic acid, glutamic acid, serine, threonine, asparagine and/or glutamine.

33. The tolerogenic peptide according to claim 31, wherein the one or more amino acid modifications comprise addition of one to four lysine residues to the N-terminus and/or C-terminus of the peptide.

34. The tolerogenic peptide according to claim 33, wherein the peptide is selected from KKKWKLSGVERKKK, KKKWKLSGVERAKKK, KKKRISNMYAMMIARRKKK, KKKKISNMYAMMIARKKKK and/or KKERRILEAKQKGFVPKK.

35. A pharmaceutical composition comprising an effective amount of tolerogenic peptide according to claim 21.

36. (canceled)

37. The tolerogenic peptide according to claim 21, wherein the peptide further comprises labelling with one or more moieties selected from: radionuclide, peptide tag, luminescent molecule, fluorescent molecule, quencher molecule, pH sensitive molecule, oxygen sensitive molecule or a combination thereof.
